(19) United States
(12) Patent Application Publication
(10) Pub. No.: US 2006/0024337 A1
(43) Pub. Date: Feb. 2, 2006

(54) Process for dissolving lipophilic compounds in aqueous solution with amphiphilic block copolymers, and cosmetic composition

(76) Inventor: Jean-Thierry Simonnet, Cachan (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/529,743

(22) PCT Filed: Oct. 16, 2003
(86) PCT No.: PCT/EP03/13050

§ 371 Date: Mar. 29, 2005

(30) Foreign Application Priority Data
Oct. 21, 2002 (FR) 0213101
Dec. 12, 2002 (US) 60/432619

(51) Int. Cl. A61K 8/81

(52) U.S. Cl. 424/401; 424/70.11

(57) ABSTRACT

The invention relates to a process for dissolving at least one lipophilic compound in at least one aqueous phase, characterized in that it comprises the step of associating the said lipophilic compound with an effective amount of at least one amphilic block copolymer comprising at least one ionic and/or at least one nonionic hydrophilic polymer block, and at least one hydrophobic polymer block.

The present invention relates to a process for dissolving lipophilic compounds with amphiphilic block copolymers. The invention also relates to novel cosmetic compositions resulting from the implementation of this process.

A large number of lipophilic molecules have very low solubilities in cosmetic solvents and have a tendency to recrystallize rapidly. Moreover, it is sought to avoid the presence of oily solvents for sensory reasons, or for reasons of compatibility in the case of dissolving two molecules with respective solvents that are incompatible with each other, or else for reasons of harmlessness if it is not desired to use solvents that can modify the barrier function of the skin.

Thus, in the case of sunscreens, in order to have substantial photoprotection, large proportions of solvent oils are necessary. This may be harmful to the final cosmetic feel of the composition containing them: oily, greasy feel.

Formulation solutions that can increase the solubility of these sunscreens in water while at the same time avoiding the use of solvents such as ethanol and isopropanol, which are harmful to the integrity of the barrier function of the skin, have thus been sought.

It is known practice to combine certain compounds with lipophilic molecules, especially DHEA, in order to improve their solubility. However, none of these compounds is a block copolymer.

It is known practice to encapsulate lipophilic compounds in micelles of block copolymers, for example poly(ethylene oxide-propylene oxide) diblock or triblock copolymers. However, these block copolymers do not satisfactorily dissolve the lipophilic compound.

It is thus seen that it is necessary to develop families of readily available copolymers, enabling a satisfactory dissolution of the lipophilic compound by encapsulation in micelles that are stable at room temperature.

The Applicant has just discovered, surprisingly, that by combining a lipophilic compound with an effective amount of at least one block amphiphilic copolymer comprising at least one ionic or nonionic hydrophilic polymer block and at least one particular hydrophobic polymer block, the lipophilic compound can be satisfactorily dissolved, thus solving the drawbacks of the prior art.

One subject of the present invention is thus a process for dissolving lipophilic compounds in an aqueous phase using block copolymers.

A subject of the invention is also a cosmetic composition comprising a lipophilic compound and a block copolymer in aqueous phase.

Other characteristics, aspects and advantages of the invention will emerge on reading the detailed description that follows.

The process for dissolving at least one lipophilic compound in aqueous phase in accordance with the invention is characterized in that it comprises the step of associating (physical combination) said lipophilic compound with an effective amount of at least one amphiphilic block copolymer comprising at least one ionic and/or at least one nonionic hydrophilic polymer block, and at least one hydrophobic polymer block obtained from at least one hydrophobic monomer chosen from:
styrene and its derivatives such as 4-butylstyrene,
vinyl acetate of formula CH₂═CH—OCOCH₃,
vinyl ethers of formula CH₂═CHOR in which R is a linear or branched, saturated or unsaturated hydrocarbon-based radical containing from 1 to 6 carbon atoms,
acrylonitrile,
vinyl chloride and vinylidene chloride,
caprolactone and caprolactam,
alkenes such as ethylene, propylene, butylene and butadiene,
alkylene oxides containing at least 4 carbon atoms and preferably from 4 to 6 carbon atoms,
silicone derivatives producing, after polymerization, silicone polymers such as polydimethylsiloxane,
hydrophobic vinyl monomers of formula (A) below: 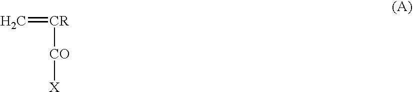
in which:
R is chosen from H, —CH₃, —C₂H₅ and —C₃H₇,
X is chosen from:
   alkyl oxides of —OR′ type in which R′ is a linear, cyclic or branched, saturated or unsaturated hydrocarbon-based radical containing from 1 to 22 carbon atoms, optionally substituted with a halogen atom (iodine, bromine, chlorine or fluorine); a sulphonic group (—SO₃⁻), a sulphate group (—SO₄⁻), a phosphate group (—PO₄H₂⁻); a hydroxyl group (—OH); a primary amine group (—NH₂); a secondary amine group (—NHR₁), a tertiary amine group (—NR₁R₂) or a quaternary amine group (—N⁺R₁R₂R₃) with R₁, R₂ and R₃ being, independently of each other, a linear or branched, saturated or unsaturated hydrocarbon-based radical containing from 1 to 22 carbon atoms, with the proviso that the sum ‘of the carbon atoms of R’+R₁+R₂+R₃ does not exceed 22; R′ may also be a perfluoroalkyl radical, preferably containing from 1 to 18 carbon atoms;
   NH₂, —NHR′ and —NR′R″ groups in which R′ and R″ are, independently of each other, linear, cyclic or branched, saturated or unsaturated hydrocarbon-based radicals containing from 1 to 22 carbon atoms, with the proviso that the total number of carbon atoms of R′+R″ does not exceed 22, R′ and R″ optionally being substituted with a halogen atom (iodine, bromine, chlorine or fluorine); a hydroxyl group (—OH); a sulphonic group (—SO₃⁻); a sulphate group (—SO₄⁻); a phosphate group (—PO₄H₂⁻); a primary amine group (—NH₂); a secondary amine group (—NHR₁), a tertiary amine group (—NR₁R₂) and/or a quaternary amine group (—N+R₁R₂R₃) with R₁, R₂ and R₃ being, independently of each other, a linear or branched, saturated or unsaturated hydrocarbon-based radical containing from 1 to 22 carbon atoms, with the proviso that the sum of the carbon atoms of R′+R″+R₁+R₂+R₃ does not exceed 22; R′ and R″ may also be perfluoroalkyl radicals, preferably containing from 1 to 18 carbon atoms.

The expression “effective amount of amphiphilic copolymer” means an amount that is sufficient for the copolymer to dissolve by itself the lipophilic compound in the aqueous phase, without recrystallization or creaming (for molecules that are oily at room temperature) of the said lipophilic compound.

The block copolymers used in the process according to the invention are especially those that can form micelles or lyotropic liquid crystal phases of lamellar, cubic (direct or inverse) or hexagonal (direct or inverse) type on contact with water. They are especially of diblock (A-B), triblock (B-A-B) or triblock (A-B-A) type, A corresponding to the ionic and/or nonionic hydrophilic polymer block and B to the hydrophobic polymer block.

The ionic hydrophilic polymer block may be obtained from water-soluble monomers or salts thereof, which may be chosen from:
(meth)acrylic acid,
acrylamido-2-methylpropanesulphonic (AMPS) acid,
styrenesulphonic acid,
vinylsulphonic acid and (meth)allylsulphonic acid,
vinylphosphonic acid,
maleic anhydride,
itaconic acid,
dimethyldiallylammonium chloride,
quaternized dimethylaminoethyl methacrylate (DMAEMA),
(meth)acrylamidopropyltrimethylammonium chloride (APTAC and MAPTAC),
methylvinylimidazolium chloride,
hydrophilic vinyl monomers of formula (A) below: 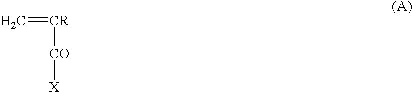
   in which:
R is chosen from H, —CH₃, —C₂H₅ and —C₃H₇,
X is chosen from:
   alkyl oxides of —OR′ type in which R′ is a linear, cyclic or branched, saturated or unsaturated hydrocarbon-based radical containing from 1 to 6 carbon atoms, substituted with at least one sulphonic group (—SO₃⁻) and/or sulphate group (—SO₄⁻) and/or phosphate group (—PO₄H₂⁻) and/or quaternary amine group (—N⁺R₁R₂R₃) with R₁, R₂ and R₃ being, independently of each other, a linear or branched, saturated or unsaturated hydro-carbon-based radical containing from 1 to 6 carbon atoms, with the proviso that the sum of the atoms of R′+R₁+R₂+R₃ does not exceed 6; the radical R′ being optionally substituted with a halogen atom (iodine, bromine, chlorine or fluorine); a hydroxyl group (—OH); a primary amine group (—NH₂); a secondary amine group (—NHR₁) or a tertiary amine group (—NR₁R₂) with R₁, R₂ being, independently of each other, a linear or branched, saturated or unsaturated hydrocarbon-based radical containing from 1 to 6 carbon atoms, with the proviso that the sum of the carbon atoms of R′+R₁+R₂ does not exceed 6;
   NH₂, —NHR′ and —NR′R″ groups in which R′ and R″ are, independently of each other, linear, cyclic or branched, saturated or unsaturated hydrocarbon-based radicals containing from 1 to 6 carbon atoms, with the proviso that the total number of carbon atoms of R′+R″ does not exceed 6, R′ and/or R″ being substituted with at least one sulphonic group (—SO₃—) and/or sulphate group (—SO₄′) and/or phosphate group (—PO₄H₂) and/or quaternary amine group (—N⁺R₁R₂R₃) with R₁, R₂ and R₃ being, independently of each other, a linear or branched, saturated or unsaturated hydrocarbon-based radical containing from 1 to 6 carbon atoms, with the proviso that the sum of the atoms of R′+R₁+R₂+R₃ does not exceed 6; the radicals R′ and/or R″ being optionally substituted with a halogen atom (iodine, bromine, chlorine or fluorine); a hydroxyl group (—OH); a primary amine group (—NH₂); a secondary amine group (—NHR₁) or a tertiary amine group (—NR₁R₂) With R₁, R₂ being, independently of each other, a linear or branched, saturated or unsaturated hydrocarbon-based radical containing from 1 to 6 carbon atoms, with the proviso that the sum of the carbon atoms of R″+R″+R₁+R₂ does not exceed 6.

The ionic hydrophilic polymer block may also be polyethyleneimine.

The ionic hydrophilic polymer block may also be obtained from hydrophobic monomers, the said hydrophobic monomers being present in an amount that is small enough for the ionic hydrophilic polymer block to be soluble in water. The hydrophobic monomers may be chosen from:
styrene and its derivatives such as 4-butylstyrene,
vinyl acetate of formula CH₂═CH—OCOCH₃,
vinyl ethers of formula CH₂═CHOR in which R is a linear or branched, saturated or unsaturated hydrocarbon-based radical containing from 1 to 6 carbon atoms,
acrylonitrile,
vinyl chloride and vinylidene chloride,
caprolactone,

hydrophobic vinyl monomers of formula (A) below:

H₂C═CR

| | |
|---|---|
| \| | (A) |

CO
|
X

in which:
—R is chosen from H, —CH₃, —C₂H₅ or —C₃H₇,
X is chosen from:
   alkyl oxides of —OR′ type in which R′ is a linear or branched, saturated or unsaturated hydrocarbon-based radical containing from 1 to 6 carbon atoms,
   NH₂, —NHR′ and —NR′R″ groups in which R′ and R″ are, independently of each other, linear or branched, saturated or unsaturated hydrocarbon-based radicals containing from 1 to 6 carbon atoms, with the proviso that the total number of carbon atoms of R′+R″ does not exceed 6.

The ionic hydrophilic polymer block is preferably chosen from (meth)acrylic acid.

Said ionic hydrophilic polymer block could be neutralized, partly or totally, by using an organic or non organic base, such as sodium, ammonium, lithium, calcium, magnesium, salts substituted by 1-4 alkyl groups containing 1-15 carbon atoms. Other suitable bases are mono-, di- or triethanolamine, aminoethylpropanediol, N-methyl glucamine, or basic aminoacids such as arginine or lysine.

The nonionic hydrophilic polymer block may be of polyoxyalkylenated type, for instance polyoxyethylene. It may also be polyvinylpyrrolidone (PVP).

The nonionic hydrophilic polymer block may also be obtained from water-monomers chosen from:
(meth)acrylamide,
N-vinylacetamide and N-methyl-N-vinylacetamide,
N-vinylformamide and N-methyl-N-vinylformamide,
N-vinyllactams comprising a cyclic alkyl group containing from 4 to 9 carbon atoms, such as N-vinylpyrrolidone, N-butyrolactam and N-vinyl-caprolactam,
vinyl alcohol of formula CH₂═CHOH,
glycidyl (meth)acrylate,
hydrophilic vinyl monomers of formula (A) below: 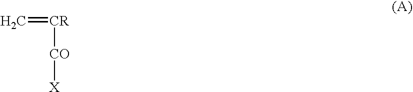
   in which:
R is chosen from H, —CH₃, —C₂H₅ and —C₃H₇,
X is chosen from:
   alkyl oxides of —OR′ type in which R′ is a linear or branched, saturated or unsaturated hydrocarbon-based radical containing from 1 to 6 carbon atoms, optionally substituted with a halogen atom (iodine, bromine, chlorine or fluorine); a hydroxyl group (—OH); a primary amine group (—NH₂); a secondary amine group (—NHR₁) or a tertiary amine group (—NR₁R₂) with R₁ and R₂ being, independently of each other, a linear or branched, saturated or unsaturated hydrocarbon-based radical containing from 1 to 6 carbon atoms, with the proviso that the sum of the carbon atoms of R′+R₁+R₂ does not exceed 6;
   NH₂, —NHR′ and —NR′R″ groups in which R′ and R″ are, independently of each other, linear or branched, saturated or unsaturated hydrocarbon-based radicals containing from 1 to 6 carbon atoms, with the proviso that the total number of carbon atoms of R′+R″ does not exceed 6, R′ and R″ optionally being substituted with a halogen atom (iodine, bromine, chlorine or fluorine); a hydroxyl group (—OH); a primary amine group (—NH₂); a secondary amine group (—NHR₁) or a tertiary amine group (—NR₁R₂) with R₁ and R₂ being, independently of each other, a linear or branched, saturated or unsaturated hydrocarbon-based radical containing from 1 to 6 carbon atoms, with the proviso that the sum of the carbon atoms of R′+R″+R₁+R₂ does not exceed 6.

The nonionic hydrophilic polymer block is preferably chosen from polyethylene oxide or polyoxyethylene (PEO) and polyvinylpyrrolidone (PVP).

Preferably, the hydrophilic polymer block is nonionic.

Preferably, the hydrophilic polymer block is an homo-polymer.

The hydrophobic polymer block is obtained from at least one of the monomers cited above. It may also be obtained from hydrophilic monomers, being present in an amount that is small enough for the hydrophobic polymer block to be hydrophobic.

The hydrophilic monomers may be chosen from the above-mentioned water-soluble monomers suitable from obtaining nonionic hydrophilic polymer block or ionic hydrophilic polymer block.

Preferably, the hydrophobic polymer block is an homo-polymer.

The hydrophobic polymer block is preferably obtained from at least one hydrophobic monomers chosen from styrene, tert-butylstyrene, methyl methacrylate, ethyl (meth)acrylate, n-butyl (meth)acrylate, tert-butyl (meth)acrylate, cyclohexyl acrylate, isobornyl acrylate, 2-ethylhexyl acrylate, ethyl perfluorooctyl acrylate, trifluoromethyl (meth)acrylate, polybutylene oxide or polyoxybutylene (POB), butadiene, ethylene, propylene and butylene.

The block copolymer is preferably chosen from the following block copolymers:
polystyrene/polyoxyethylene
polymethyl methacrylate/polyoxyethylene
polybutyl methacrylate/polyoxyethylene
polyoxybutylene/polyoxyethylene
polycaprolactone/polyoxyethylene
polyethylene/polyoxyethylene
polyoxyethylene/polyoxybutylene/polyoxyethylene.

The molecular weight of the block copolymer may be between 1 000 and 500 000, preferably from 2000 to 100 000.

The molecular weight of the hydrophilic block may be between 600 and 300 000, preferably from 1200 to 60 000.

The molecular weight of the hydrophobic block may be between 400 and 200 000, preferably from 800 to 40 000.

The weight ratio A/B between the hydrophilic block A and the hydrophobic block B ratio may be between 1/100 and 50/1.

In addition, the weight concentration ratio between the lipophilic compound and the amphiphilic block copolymer, used according to the invention is generally between 0.005 and 0.5 and preferably between 0.005 and 0.2.

The lipophilic compounds are preferably those with a solubility in water of less than 0.2% by weight. The measurement is performed by UV spectrometry or HPLC on an aqueous solution, prepared beforehand, of from 20 to 80° C. for 2 hours, followed by cooling to 25° C. The assay is performed 24 hours after this preparation on the supernatant obtained after centrifuging the suspension to remove all crystals, or on the pellet in the case of solubilization of oily molecules, the centrifugation resulting in creaming of the portion that is not encapsulated in the micelles.

In order for the gain in solubility to be satisfactory, the solubility in the aqueous phase of the lipophilic compound encapsulated in the block copolymer must be at least 15 times greater than the “natural” solubility of the lipophilic compound in water.

Examples of lipophilic compounds that may be mentioned include emollients, anti-inflammatory agents, antibacterial agents, antifungal agents, antiviral agents, anti-seborrhoeic agents, antiacne agents, keratolytic agents, antihistamines, anesthetics, cicatrizing agents, pigmentation modifiers, tanning accelerators, artificial tanning agents, liporegulators, anti-ageing and anti-wrinkle agents, refreshing agents, vascular protectors, insect repellants, deodorants, antidandruff agents, agents for preventing hair loss, essential oils, which may be chosen especially from eucalyptus oil, lavandin oil, lavender oil, vetiver oil, Litsea cubeba oil, lemon oil, sandalwood oil, rosemary oil, camomile oil, savory oil, nutmeg oil, cinnamon oil, hyssop oil, caraway oil, orange oil, geraniol oil and cade oil, fragrances, sunscreens, antioxidants, free-radical scavengers and moisturizers.

Examples of lipophilic compounds that may also be mentioned include vitamins such as vitamin A (retinol) or esters thereof, vitamin E or esters thereof such as tocopheryl acetate, vitamin D or derivatives thereof and vitamin F or derivatives thereof; carotenes such as β-carotene and derivatives thereof such as lycopene, and salicylic acid derivatives, especially those described in documents FR-A-2 581 542, EP-A-378 936 and EP-A-570 230.

The salicylic acid derivatives may be chosen especially from the derivatives of formula 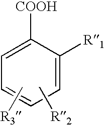
in which:
R″₁ represents a hydroxyl radical or an ester of formula —O—CO—R″₄ in which
R″₄ is a saturated or unsaturated aliphatic radical containing from 1 to 26 carbon atoms and preferably from 1 to 18 carbon atoms, or an amine or thiol function optionally substituted with an alkyl radical containing from 1 to 18 carbon atoms and preferably from 1 to 12 carbon atoms,
R″₂ and R″₃, independently of each other, are in one of the positions 3, 4, 5 or 6 on the benzene nucleus and represent, independently of each other, a hydrogen atom or a radical: —(O)ₙ—(CO)ₘ—R″₅ in which n and m, independently of each other, are each an integer equal to 0 or 1, on condition that R″₂ and R″₃ are not simultaneously hydrogen atoms; and R″₅ represents a hydrogen, a saturated, linear, branched or cyclized aliphatic radical containing from 1 to 18 carbon atoms, an unsaturated radical containing from 3 to 18 carbon atoms, bearing one to nine conjugated or non-conjugated double bonds, the radicals possibly being substituted with at least one substituent chosen from halogen atoms (fluorine, chlorine, bromine or iodine), trifluoromethyl radicals, hydroxyl radicals in free form or esterified with an acid containing from 1 to 6 carbon atoms, or carboxyl radicals in free form or esterified with a lower alcohol containing from 1 to 6 carbon atoms, or an aromatic radical containing from 6 to 10 carbon atoms.

Preferably, the salicylic acid derivative is such that R″₅ represents a saturated aliphatic radical containing from 3 to 15 carbon atoms.

Preferably, the salicylic acid derivative is such that R″₁ represents a hydroxyl radical.

Preferably, the salicylic acid derivative is such that R″₅ is in position 5 of the benzene nucleus and R″₂ represents a hydrogen atom.

According to one preferred embodiment of the invention, the salicylic acid derivative is chosen from the 5-n-octanoylsalicylic, 5-n-decanoylsalicylic, 5-n-dodecanoylsalicylic, 5-n-octylsalicylic, 5-n-heptyloxysalicylic, 4-n-heptyloxysalicylic, 5-tert-octylsalicylic, 3-tert-butyl-5-methylsalicylic, 3-tert-butyl-6-methylsalicylic, 3,5-diisopropylsalicylic, 5-butoxysalicylic, 5-octyloxysalicylic, 5-propanoylsaiicylic, 5-n-hexadecanoylsalicylic, 5-n-oleoylsalicylic and 5-benzoylsalicylic derivatives, monovalent and divalent salts thereof, and mixtures thereof.

When it is a sunscreen, the lipophilic compound may be chosen from anthranilates; cinnamic derivatives; dibenzoylmethane derivatives; salicylic derivatives; camphor derivatives; triazine derivatives, preferably 1,3,5-triazine derivatives such as those described in patent applications U.S. Pat. Nos. 4,367,390, 4,724,137, EP 863 145, EP 517 104, EP 570 838, EP 796 851, EP 775 698, EP 878 469, EP 933 376, EP 507 691, EP 507 692, EP 790 243 and EP 944-624; benzophenone derivatives; diphenylacrylate derivatives; benzotriazole derivatives; benzalmalonate derivatives; benzimidazole derivatives; imidazolines; bis-benzazolyl derivatives as described in patents EP 669 323 and U.S. Pat. No. 2,463,264; p-aminobenzoic acid (PABA) derivatives; methylene-bis(hydroxyphenylbenzotriazole) derivatives as described in patent applications U.S. Pat. Nos. 5,237,071, 5,166,355, GB 2303546, DE 19726184 and EP 893 119; screening polymers and screening silicones such as those described especially in patent application WO 93/04665; dimers derived from α-alkylstyrene, such as those described in patent application DE 198 55 649; 4,4-diarylbutadienes as described in patent applications EP 0 967 200 and DE 197 55 649, and mixtures thereof.

One preferential 1,3,5-triazine is 2,4,6-tris[p-(2′-ethylhexyl-1-oxycarbonyl)anilino]-1,3,5-triazine, which is a UV-B-active screening agent that is known per se, which is in a solid form and which is sold especially under the trade name “Uvinul T150” by the company BASF. This product corresponds to the following formula: 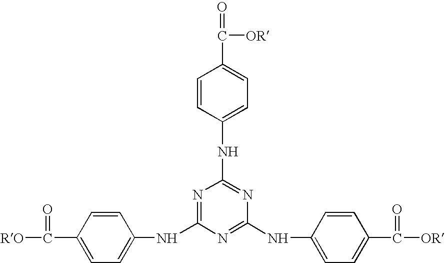
in which R′ denotes a 2-ethylhexyl radical.

Other 1,3,5-triazine derivatives used according to the invention that are particularly preferred are 2-[(p-(tert-butylamido)anilino]-4,6-bis[(p-(2′ethylhexyl-1′-oxycarbonyl)anilino]-1,3,5-triazine, 2,4-bis{[4-2-ethylhexyloxy]-2-hydroxy]phenyl}-6-(4-methoxyphenyl)-1,3,5-triazine, 2,4,6-tris(diisobutyl 4′-aminobenzalmalonate)-s-triazine, and mixtures thereof.

Among the dibenzoylmethane derivatives, it is most particularly preferred to use 4-(tert-butyl)-4′-methoxydibenzoylmethane, or butylmethoxydibenzoyl-methane, sold especially under the trade name “Parsol® 1789” by the company Hoffmann Laroche.

According to the present invention, the lipophilic compounds may also be chosen from:
(1) dehydroepiandrosterone (DHEA) and its precursors and chemical and biological derivatives, with the exception of cholesterol and its esters and plant sterols such as phytosterols and sitosterols, and esters thereof; dehydroepiandrosterone is a natural steroid produced essentially by the adrenal glands, corresponding to the formula

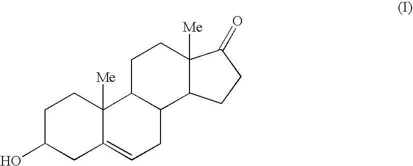

It is known for its anti-ageing properties associated with its capacity to promote epidermal keratinization (JP-07 196 467) and to combat osteoporosis (U.S. Pat. No. 5,824,671), or in the treatment of dry skin, on account of its ability to increase the endogenous production and secretion of sebum and to reinforce the skin's barrier effect (U.S. Pat. No. 4,496,556). It has also been proposed to use DHEA sulphate against alopecia (JP-60 142 908) and to treat various signs of ageing such as wrinkles, loss of radiance of the skin and slackening of the skin (EP-0 723 775).

The DHEA that may be used according to the invention is available, for example, from the companies Sigma and Akzo Nobel.

The expression “DHEA precursor” means the immediate biological precursors thereof and also the chemical precursors thereof. Examples of biological precursors are pregnenolone, 17α-hydroxypregnenolone, 5-androstenediol, 17α-hydroxypregnenolone sulphate and 5-androstenediol sulphate. Examples of chemical precursors are sapogenins such as diosgenin (spirost-5-ene-3-beta-ol), hecogenin, smilagenin and sarsapogenin, and also natural extracts containing them, in particular fenugreek and extracts of Dioscorea plants such as wild yam root.

The expression “DHEA derivatives” means both the metabolic derivatives thereof and the chemical derivatives thereof. Metabolic derivatives which may be mentioned in particular include 7α-hydroxy-DHEA, 7-keto-DHEA, 5-androstene-3β, 17β-diol (or adiol), 5-androstene-3β,17β-diol sulphate and 4-androstene-3,17-dione, although this list is not limiting. Chemical derivatives that may be mentioned in particular include salts, in particular water-soluble salts such as DHEA sulphate, esters such as the hydroxycarboxylic acid esters of DHEA described in U.S. Pat. No. 5,736,537 or other esters such as DHEA salicylate, acetate, valerate and enanthate;
(2) pentacyclic triterpene acids such as ursolic acid and oleanolic acid.

They are present in plants such as rosemary. They are frequently used in pharmaceutical compositions for their numerous therapeutic properties, and especially for their anti-inflammatory, hepato-protective, diuretic, analgesic and antimicrobial properties, their inhibitory properties on certain enzymatic activities, and their antitumour properties. In the cosmetic field, ursolic acid is described, for example, as a constituent of an antiperspirant composition (FR A 2 541 895) and as an inhibitor of the activity of tyrosinase, a key enzyme in melanin synthesis (JP-58157307).

(3) hydroxystilbenes, which are compounds corresponding to the general formula: 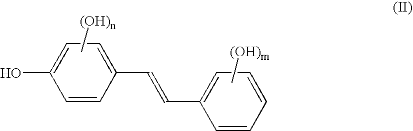
in which n is an integer between 1 and 4 inclusive and m is an integer between 1 and 5 inclusive. This formula includes the cis and trans compounds. According to the present invention, the term “hydroxystilbene” also covers the hydroxyalkyl derivatives of the compounds of formula (II).

Hydroxystilbenes are compounds that are found in the natural state in plants of the spermatophyte class and in particular in vine. In the cosmetic field, hydroxystilbenes are used, inter alia, as depigmenting agents (JP-87-192 040) or anti-ageing agents (FR-2 777 186).

Among the hydroxystilbenes that may be mentioned are mono-, di-, tri-, tetra-, penta-, hexa-, hepta-, octa- and nonahydroxystilbenes, or hydroxyalkyl derivatives thereof.

According to the invention, the hydroxystilbenes may be used alone or as mixtures of any nature and may be of natural or synthetic origin. The hydroxystilbenes that may be used according to the invention are chosen from:
4′-hydroxystilbene,
2′,4′-dihydroxystilbene,
3′,4′-dihydroxystilbene,
4,4-dihydroxystilbene,
2′,4′,4-trihydroxystilbene,
3′,4′,4-trihydroxystilbene,
2,4,4′-trihydroxystilbene,
3,4,4′-trihydroxystilbene,
3,5,4′-trihydroxystilbene,
2′,3,4-trihydroxystilbene,
2,3′,4-trihydroxystilbene,
2′,2,4′-trihydroxystilbene,
2,4,4′,5′-tetrahydroxystilbene,
2′,3,4′,5-tetrahydroxystilbene,
2,2′,4,4′-tetrahydroxystilbene,
3,3′,4′,5-tetrahydroxystilbene,
2,3′,4,4′-tetrahydroxystilbene,
3,3′,4,4′-tetrahydroxystilbene,
3,3′,4′,5,5′-pentahydroxystilbene,
2,2′,4,4′,6-pentahydroxystilbene,
2,3′,4,4′,6-pentahydroxystilbene,
2,2′,4,4′,6,6′-hexahydroxystilbene.

Among these compounds, resveratrol (3,5,4′-trihydroxystilbene) is particularly preferred since it is naturally present in the skin of grape seeds and in wine. In this regard, the review by Soleas and collaborators (Clinical Biochemistry, vol. 30, No. 2, pages 91-113, 1997), which perfectly summarizes the state of knowledge regarding this compound and hydroxystilbenes, may be consulted.
(4) isoflavonoids, a sub-class of flavonoids, which are formed from a 3-phenylchroman skeleton that is oxidized to a greater or lesser extent and that may bear various substituents. The term “isoflavonoid” covers several classes of compounds, among which mention may be made of isoflavones, isoflavanones, rotenoids, pterocarpans, isoflavans, isoflavan-3-enes, 3-arylcoumarins, 3-aryl-4-hydroxycoumarins, coumestanes, coumarono-chromones or 2-arylbenzofurans. A full review of isoflavonoids, their sources and methods of analysis has been published in “The Flavonoids”, Harbone editor (1988), chapter 5 entitled “Isoflavonoids” by P. M. Dewick, pages 125-157.

The isoflavonoids used according to the invention have a solubility in water at room temperature (25° C.) of less than 0.01% and may be of natural origin, that is to say extracts of an element of natural origin, usually a plant, or may have been obtained by chemical synthesis. Isoflavonoids of natural origin are preferred.

An example of an isoflavonoid of natural origin that may be mentioned is genistin.

A preferred sub-class of isoflavonoids is that of isoflavones, covering both the aglycone forms (daidzein, genistein and glycitein) and the glycosyl forms (daidzin, genistin, glycitin).

Processes for preparing isoflavones are described in particular in patents and patent applications WO 95/10530, WO 95/10512, U.S. Pat. No. 5,679,806, U.S. Pat. No. 5,554,519, EP 812 837 and WO 97/26269.

Isoflavones are known in particular as antioxidants, for their free-radical-scavenging and depigmenting properties, and also for their capacity to inhibit the activity of sebaceous glands (DE-44 32 947). They have also been described as agents capable of preventing signs of ageing of the skin (JP 1-96106).
(5) aminophenol derivatives of formula 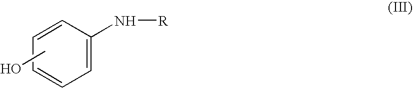
   in which R is a radical corresponding to one of the formulae (i), (ii) and (iii) below
   —CO—NR₁R₂ (i)
   —CO—O—R₃ (ii)
   —SO₂—R₃ (iii)
   in which R₁ represents a hydrogen atom or an optionally hydroxylated, saturated or unsaturated, linear or branched C₁₋₆ alkyl radical,
R₂ represents a hydrogen atom or a radical chosen from optionally hydroxylated, saturated or unsaturated, linear, cyclic or branched C₁₂ to C₃₀ alkyl radicals, and
R₃ represents a radical chosen from saturated or unsaturated, linear, branched or cyclic C₁₂ to C₃₀ alkyl radicals, including fused polycyclic radicals, which are optionally hydroxylated.

Examples of lipophilic compounds that are preferred according to the present invention which may be mentioned include dehydroepiandrosterone (DHEA), DHEA sulphate, 7α-hydroxy-DHEA, 7-keto-DHEA, prednisolone, prednisone, progesterone, pregnenolone, testosterone, diosgenin, hecogenin, ursolic acid, oleanolic acid, resveratrol (=3,5,4′-trihydroxy-stilbene) and N-cholesteryloxycarbonyl-4-aminophenol, and isoflavonoids whose solubility in water at room temperature (25° C.) is less than 0.01%.

A subject of the invention is also a cosmetic composition comprising at least one aqueous phase, at least one lipophilic compound and an effective amount of at least one block amphiphilic copolymer as above defined.

The lipophilic compound may be present in the composition in weight contents of between 0.001% and 10% of the total weight of the composition.

The method for implementing the block copolymer(s) and the lipophilic compound(s) to be combined will be chosen by a person skilled in the art as a function of the chemical nature of the polymer. Examples that will be mentioned include nanoprecipitation via a water-miscible solvent, dialysis or direct hydration of the pre-combined block copolymer/lipophilic compound mixture. Nanoprecipitation consists in co-dissolving, in a water-miscible organic solvent with a boiling point below that of water, the block copolymer(s) and the lipophilic compound to be encapsulated and in introducing this organic solution more or less quickly into an aqueous phase, with stirring (for example using a magnetic bar, paddles or a turbomixer). The micelles or particles form instantaneously. The solvent is then evaporated off.

The dialysis method consists in co-dissolving, in a water-miscible to sparingly water-miscible organic solvent with a boiling point higher than that of water, the block copolymer(s) and the lipophilic compound to be encapsulated. This solution is introduced into a dialysis bag and dialysed against water. The dialysis water is regularly renewed, until the solvent has been completely dialysed. The same method may be envisaged with surfactants, for instance octyl glucoside, instead of solvent. It is a concentrated aqueous solution of surfactants that dissolves the copolymer(s) and the lipophilic compound to give a clear solution. This solution is then dialysed until the surfactant is completely removed.

Another method, known as the Bangham method, which is commonly used for making liposomes, may be used. This consists in dissolving, in an organic solvent, the block copolymer(s) and the lipophilic compound to be encapsulated, and then evaporating off the solvent so as to obtain a uniform mixture of the block copolymer(s) and of the lipophilic compound to be encapsulated. A rotary evaporator may be used in the laboratory, an atomizer may be used industrially, or any other method for evaporating a solvent may be used. This mixture may then be hydrated directly, with stirring, using an aqueous solution.

In general, any method known to those skilled in the art for synthesizing liposomes and nanoparticles may be used.

According to one embodiment of the invention, the block amphiphilic copolymer is the sole solvent for the lipophilic compound in the composition.

The compositions according to the invention may also contain pigments or nanopigments (mean size of the primary particles: generally between 5 nm and 100 nm and preferably between 10 and 50 nm) of coated or uncoated metal oxides, for instance nanopigments of titanium oxide (amorphous or crystallized in rutile and/or anatase form), iron oxide, zinc oxide, zirconium oxide or cerium oxide, which are photoprotective agents that are well known per se, acting by physically blocking (reflection and/or scattering) UV radiation. Standard coating agents are, moreover, alumina and/or aluminium stearate. Such coated or uncoated metal oxide nanopigments are described in particular in patent applications EP-A-0 518 772 and EP-A-0 518 773.

The compositions in accordance with the present invention may also contain standard cosmetic adjuvants chosen especially from fatty substances, organic solvents, ionic or nonionic thickeners, softeners, free-radical scavengers, opacifiers, stabilizers, emollients, silicones, α-hydroxy acids, antifoams, moisturizers, vitamins, preserving agents, surfactants, fillers, sequestering agents, polymers, propellants, acidifying or basifying agents, dyes or any other ingredient usually used in cosmetics and/or dermatology, in particular to manufacture antisun compositions in the form of emulsions.

The fatty substances may consist of an oil or a wax or mixtures thereof.

The term “oil” means a compound that is liquid at room temperature. The term “wax” means a compound that is solid or substantially solid at room temperature, and which has a melting point generally above 35° C.

Oils that may be mentioned include mineral oils (vaseline); plant oils (sweet almond oil, macadamia oil, blackcurrant seed oil or jojoba oil); synthetic oils, for instance perhydrosqualene, fatty alcohols, fatty acids or fatty esters (for instance the C₁₂-C₁₅ alcohol benzoates sold under the name “Finsolv TN” by the company Finetex, octyl palmitate, isopropyl lanolate, triglycerides including those of capric/caprylic acids), ethoxylated or propoxylated fatty esters and ethers; silicone oils (cyclomethicone preferably containing 4 or 5 silicon atoms, and polydimethylsiloxane or PDMS) or fluoro oils, and polyalkylenes.

Waxy compounds that may be mentioned include paraffin, carnauba wax, beeswax and hydrogenated castor oil.

Among the organic solvents that may be mentioned are lower alcohols and polyols.

The thickeners may be chosen especially from crosslinked polyacrylic acids, modified or unmodified guar gums and celluloses, such as hydroxypropyl guar gum, methylhydroxyethylcellulose or hydroxypropylmethylcellulose, and silicone gums, for instance a polydimethylsiloxane derivative.

The compositions of the invention may be prepared according to the techniques that are well known to those skilled in the art, in particular those intended for preparing emulsions of the oil-in-water or water-in-oil type.

These compositions may be In particular in the form of a dispersion, especially an aqueous dispersion, a simple or complex emulsion (O/W, W/O, O/M/O or W/O/W emulsion) such as a cream, a milk or a cream-gel, a powder or a solid tube, and may optionally be packaged as an aerosol and may be in the form of a mousse or a spray.

When it is an emulsion, the aqueous phase of the said emulsion may comprise a nonionic vesicular dispersion prepared according to known processes (Bangham, Standish and Watkins, J. Mol. Biol. 13, 238 (1965), FR 2315991 and FR 2416008).

The cosmetic compositions according to the invention may be used as compositions for protecting the human epidermis or the hair against ultraviolet rays, as antisun compositions or as makeup products.

When the cosmetic compositions according to the invention are used for protecting the human epidermis against UV rays or for antisun compositions, they may be in the form of a suspension or a dispersion in fatty substances, in the form of a nonionic vesicular dispersion or in the form of an emulsion, preferably of oil-in-water type, such as a cream or a milk, or in the form of an ointment, a gel, a solid tube, a stick, an aerosol mousse or a spray.

When the cosmetic compositions according to the invention are used for protecting the hair, they may be in the form of a shampoo, a lotion, a gel, an emulsion or a nonionic vesicular dispersion. They may constitute, for example, a rinse-out composition, to be applied before or after shampooing, before or after dyeing or bleaching, or before, during or after permanent-waving or relaxing the hair, a styling or treating lotion or gel, a blow-drying or hairsetting lotion or gel, or a composition for permanent-waving, relaxing, dyeing or bleaching the hair.

When the cosmetic compositions according to the invention are used as makeup products for the eyelashes, the eyebrows or the skin, such as an epidermal treatment cream, a foundation, a tube of lipstick, an eyeshadow, a makeup rouge, a mascara or an eyeliner, they may be in solid or pasty, anhydrous or aqueous form, for instance oil-in-water or water-in-oil emulsions, nonionic vesicular dispersions or suspensions.

The examples that follow are intended to illustrate the invention.

### Example 1

The block copolymer is dissolved in dichloromethane with excess DHEA.

The solvent is evaporated off under reduced pressure using a rotary evaporator. The film formed is then hydrated with distilled water, with stirring, at a temperature of between 20 and 80° C., for 2 hours. The polymer content in the water is of the order of one percent. 24 hours later, this suspension is centrifuged so as to separate out the DHEA not encapsulated in the polymer micelles.

The supernatant is taken up and then assayed by HPLC.

| | | | DHEA/ | | |
|---|---|---|---|---|---|
| | | | polymer | | |
| Chemical | | Solubility | mass | | A/B or |
| nature | Solubility | gain | ratio | MW | A/B/A |
| Water | 0.001 | | | | |
| PS-POE | 0.061 | 61 | 1.22% | 2000 | 1000/1000 |
| PS-POE | 0.036 | 36 | 0.72% | 4000 | 1000/3000 |
| Poly(methyl | 0.055 | 55 | 1.10% | 2000 | 1000/1000 |
| methacrylate)/ | | | | | |
| POE | | | | | |
| Poly(methyl | 0.023 | 23 | 0.46% | 4000 | 1000/3000 |
| methacrylate)/ | | | | | |
| POE | | | | | |
| Poly(butyl | 0.052 | 25 | 1.04% | 2000 | 1000/1000 |
| methacrylate)/ | | | | | |
| POE | | | | | |
| Poly(butyl | 0.034 | 34 | 0.68% | 4000 | 1000/3000 |
| methacrylate)/ | | | | | |
| POE | | | | | |
| POE-POB- | 0.031 | 31 | 0.62% | 9000 | 3500/2000/ |
| POE | | | | | 3500 |
| PS-POE | 0.01 | 10 | 0.20% | 10 600 | 3600/7000 |
| PS-POE | 0.06 | 60 | 1.20% | 9000 | 3900/5100 |
| PS-POE/PS- | 0.02 | 20 | 0.40% | | 3600/7000/ |
| POE | | | | | 12200/23900 |
| (90/10) | | | | | |
| PS-POE/PS- | 0.08 | 80 | 1.60% | | 3900/5100/ |
| POE | | | | | 12200/23900 |
| (90/10) | | | | | |
| POE-POP- | 0.005 | 5 | 0.10% | 14 600 | 128/54/128 |
| POE (com- | | | | | |
| parative) | | | | | |
| POE-POP- | 0.002 | 2 | 0.04% | 8400 | 75/30/75 |
| POE (com- | | | | | |
| parative) | | | | | |
| POE-POP- | 0.003 | 1 | 0.06% | 2200 | |
| POE (com- | | | | | |
| parative) | | | | | |
| POE-POP- | 0.002 | 2 | 0.04% | 4700 | 46/16/46 |
| POE (com- | | | | | |
| parative) | | | | | |

PS polystyrene
POE polyoxyethylene
POP polyoxypropylene
POB polyoxybutylene

The solubility gain is calculated relative to the “natural” solubility of the lipophilic compound in water.

It is found that the POE-POP-POE block copolymers do not make it possible to dissolve the DHEA sufficiently (less than fifteen times).

On the other hand, the polymers according to the invention, for instance PS-POE, methyl methacrylate-POE, butyl methacrylate-POE or POE-POB-POE, make it possible to improve the solubility of DHEA in water, up to 80 times.

### Example 2

The block polymer is dissolved in dichloromethane with excess Parsol® 1789. The solvent is evaporated off under reduced pressure using a rotary evaporator. The film formed is then hydrated with distilled water, with stirring, at a temperature of between 20 and 80° C., for 2 hours. The polymer content in the water is of the order of one percent. 24 hours later, this suspension is centrifuged in order to separate out the Parsol® 1789 that has not been encapsulated in the polymer micelles. The supernatant, which should not contain any crystals that may be identified using an optical microscope in polarized light, is taken up and then assayed by HPLC.

| | | | Parsol | |
|---|---|---|---|---|
| | | | 1789/ | |
| Chemical | Solubility | Solubility | polymer | A/B or |
| nature | mg/ml | gain | mass ratio | A/B/A |
| Water | 0.001 | | | |
| PS-POE | 0.686 | 686 | 3.43% | 1000/1000 |
| PS-POE | 0.34 | 340 | 1.70% | 1000/3000 |
| Methyl meth- | 0.862 | 862 | 4.31% | 1000/1000 |
| acrylate/POE | | | | |
| Methyl meth- | 0.43 | 430 | 2.15% | 1000/3000 |
| acrylate/POE | | | | |
| Butyl meth- | 1.008 | 1008 | 5.04% | 1000/1000 |
| acrylate/POE | | | | |
| Butyl meth- | 0.475 | 475 | 2.37% | 1000/3000 |
| acrylate/POE | | | | |
| POE-POB- | 0.230 | 230 | 1.18% | 3500/2000/ |
| POE | | | | 3500 |
| PDMS/OE/OP | 0.171 | 171 | 0.85% | PDMS/OE/ |
| | | | | OP |
| PDMS/OE/OP | 0.186 | 186 | 0.93% | PDMS/OE/ |
| | | | | OP |
| PS-POE | 0.171 | 171 | 0.85% | 3600/7000 |
| PS-POE | 0.185 | 185 | 0.92% | 3900/5100 |
| PS-POE/PS- | 0.53 | 530 | 2.65% | 3600/7000/ |
| POE | | | | 12200/23900 |
| (90/10) | | | | |
| PS-POE/PS- | 0.453 | 453 | 2.26% | 3900/5100/ |
| POE | | | | 12200/23900 |
| (90/20) | | | | |
| PS-POE | 0.529 | 529 | 2.64% | 2300/3100 |
| P(capro- | 0.316 | 316 | 1.58% | 800/5000 |
| lactone)-POE | | | | |
| P(capro- | 0.635 | 635 | 3.17% | 1370/5000 |
| lactone)-POE | | | | |
| PE-POE | 0.164 | 164 | 0.82% | 5000/5900 |
| PE-POE | 0.124 | 124 | 0.62% | 4800/4800 |
| POE-POP- | 0.006 | 6 | 0.03% | 75/30/75 |
| POE (com- | | | | |
| parative) | | | | |

PS polystyrene
POE polyoxyethylene
POP polyoxypropylene
POB polyoxybutytene

The solubility gain is calculated relative to the “natural” solubility of the lipophilic compound in water.

Surprisingly, in this case also, the block copolymers according to the invention dissolve the lipophilic compound better than do the POE-POP-POE block copolymers.

### Example 3

### Oil-in-water Emulsion

| Oily phase: | | |
|---|---|---|
| Diglyceryl monostearate | | 2.0% |
| PEG-20 stearate | | 1.5% |
| Disodium N-stearoyl-L-glutamic acid | | 0.5% |
| (Acyl glutamate HS 21 from Ajinomoto) | | |
| Liquid petroleum jelly | | 3% |
| Petroleum jelly | | 1% |
| Stearyl heptanoate | | 3% |
| Apricot kernel oil | | 5% |
| Hydrogenated polyisobutene | | 5% |
| Isocetyl palmitate | | 2% |
| Volatile silicone | | 5% |
| Vitamin E | | 0.5% |
| Preserving agent | | 0.3% |
| Aqueous phase 1 | | |
| Glycerol | | 5% |
| Preserving agents | | 1% |
| Distilled water | qs | 100% |
| Aqueous phase 2 | | |
| Carbomer | | 0.4% |
| Distilled water | | 15% |
| Preserving agents | | 0.1% |
| Triethanolamine | | 0.4% |
| Aqueous phase 3 | | |
| Aqueous suspension of ME 10—10 | | 10% |
| (methyl methacrylate/POE 1000/1000) | | |
| at 20% in water, containing 8% Parsol ® 1789 | | |

Procedure: The aqueous phase 1 is introduced at 60° C. into the oily phase at 60° C., with very vigorous stirring. The temperature and the stirring are maintained for 30 minutes. The suspension is then cooled to room temperature. The aqueous phase 2 is then dispersed using a non-shear disperser. The aqueous phase 3 (micellar suspension) is then introduced with gentle stirring.

### Example 4

### Oil-in-water Emulsion

Oily phase:

| | | |
|---|---|---|
| Diglyceryl monostearate | | 2.0% |
| PEG-20 stearate | | 1.5% |
| Disodium N-stearoyl-L-glutamic acid | | 0.5% |
| (Acyl glutamate HS 21 from Ajinomoto) | | |
| Liquid petroleum jelly | | 3% |
| Petroleum jelly | | 1% |
| Stearyl heptanoate | | 3% |
| Apricot kernel oil | | 5% |
| Hydrogenated polyisobutene | | 5% |
| Isocetyl palmitate | | 2% |
| Volatile silicone | | 5% |
| Vitamin E | | 0.5% |
| Preserving agent | | 0.3% |
| Aqueous phase 1 | | |
| Glycerol | | 5% |
| Preserving agents | | 1% |
| Distilled water | qs | 100% |
| Aqueous phase 2 | | |
| Carbomer | | 0.4% |
| Distilled water | | 15% |
| Preserving agents | | 0.1% |
| Triethanolamine | | 0.4% |
| Aqueous phase 3 | | |
| Aqueous suspension of PS-POE (1000—1000) at 20% in | | 10% |
| water, containing 1% DHEA relative to the polymer | | |

Procedure: The aqueous phase I is introduced at 60° C. into the oily phase at 60° C., with very vigorous stirring. The temperature and the stirring are maintained for 30 minutes. The suspension is then cooled to room temperature. The aqueous phase 2 is then dispersed using a non-shear disperser. The aqueous phase 3 (micellar suspension) is then introduced with gentle stirring.

### Example 5

An essential oil of lavender is dissolved, forming a micellar suspension of butyl methacrylate-POE (1000-1000)

| Aqueous phase 1 | | |
|---|---|---|
| Butyl methacrylate-POE (1000—1000) | | 10% |
| Essential oil of lavender | | 0.15% |
| Distilled water | | 80% |
| Aqueous phase 2 | | |
| Glycerol | | 3% |
| Preserving agents | | 0.3% |
| Distilled water | qs | 100% |

Procedure: The aqueous phase I is heated to 60° C. and is then mixed with stirring with the aqueous phase 2, which has itself been preheated to 60° C. The temperature and the stirring are maintained for 30 minutes. The composition is then cooled to room temperature.

1-40. (canceled)

41. A process for dissolving at least one lipophilic compound in an aqueous phase, comprising:
mixing the lipophilic compound with at least one block amphiphilic copolymer, and
mixing the mixture of the dissolved hydrophilic compound and the amphiphilic copolymer with water or an aqueous phase of a multiphase composition,
wherein the amphiphilic copolymer comprises at least one of an ionic or a nonionic hydrophilic polymer block and at least one hydrophobic polymer block wherein the hydrophobic polymer block comprises polymerized monomer units of at least one selected from the group consisting of:
styrene, derivatives thereof, 4-butylstyrene,
vinyl acetate of formula CH₂═CH—OCOCH₃, a vinyl ether of formula CH₂═CHOR in which R is a linear or branched, saturated or unsaturated hydrocarbon radical containing from 1 to 6 carbon atoms,
acrylonitrile,
vinyl chloride, vinylidene chloride,
caprolactone, caprolactam,
an alkene, ethylene, propylene, butylene, butadiene,
an alkylene oxide containing at least 4 carbon atoms, an alkylene oxide containing from 4 to 6 carbon atoms,
a silicon-containing polymerizable monomer capable of forming a polysiloxane,
a hydrophobic vinyl monomer of formula (A): 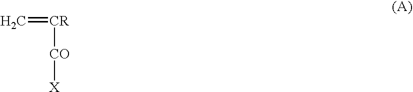
in which:
R is at least one of H, —CH₃, —C₂H₅ or —C₃H₇,
X is at least one of:
an alkyl oxide of formula —OR′ in which R′ is a linear or branched, saturated or unsaturated hydrocarbon radical containing from 1 to 22 carbon atoms, optionally substituted with at least one of a halogen atom selected from the group consisting of iodine, bromine, chlorine and fluorine; a sulphonic group (—SO₃⁻); a sulphate group (—SO₄⁻); a phosphate group (—PO₄H₂⁻); a hydroxyl group (—OH); a primary amine group (—NH₂); a secondary amine group (—NHR₁), a tertiary amine group (—NHR₂) or a quaternary amine group (—N⁺R₁R₂R₃) wherein R₁, R₂ and R₃ are, independently of each other, a linear or branched, saturated or unsaturated hydrocarbon radical containing from 1 to 22 carbon atoms, and wherein the sum of the carbon atoms of R′+R₁+R₂+R₃ does not exceed 22, or
an —NH₂, —NHR′ or —NR′R″ group in which R′ and R″ are, independently of each other, linear or branched, saturated or unsaturated hydrocarbon radicals containing from 1 to 22 carbon atoms, wherein the total number of carbon atoms of R′+R″ does not exceed 22, wherein R′ and R″ may optionally be substituted with a at least one of halogen atom selected from the group consisting of iodine, bromine, chlorine and fluorine; a hydroxyl group (—OH); a sulphonic group (—SO₃⁻); a sulphate group (—SO₄⁻); a phosphate group (—PO₄H₂⁻); a primary amine group (—NH₂); a secondary amine group (—NHR₁); a tertiary amine group (—NR₁R₂) or a quaternary amine group (—N⁺R₁R₂R₃); wherein R₁, R₂ and R₃ are, independently of each other, a linear or branched, saturated or unsaturated hydrocarbon radical containing from 1 to 22 carbon atoms, wherein the sum of the carbon atoms of R′⁺R″⁺R₁+R₂+R₃ does not exceed 22, wherein R′ and R″ may optionally be perfluoroalkyl radicals having from 1 to 18 carbon atoms.

42. The process according to claim 41, wherein the molecular weight of the block copolymer is between 1,000 and 100,000.

43. The process according to claim 41, wherein the weight ratio of the ionic or nonionic hydrophilic polymer block to the hydrophobic polymer block is between 1/100 and 50/1.

44. The process according to claim 41, wherein the weight concentration ratio between the lipophilic compound and the block amphiphilic copolymer is between 0.005 and 0.5.

45. The process according to claim 41, wherein the hydrophobic polymer block comprises polymerized units of one or more hydrophobic monomers selected from the group consisting of methyl methacrylate, ethyl methacrylate, n-butyl (meth)acrylate, tert-butyl (meth)acrylate, cyclohexyl acrylate, isobornyl acrylate, 2-ethylhexyl acrylate, ethyl perfluorooctyl acrylate and trifluoromethyl (meth)acrylate.

46. The process according to claim 41, wherein the amphiphilic copolymer comprises an ionic hydrophilic polymer block comprising one or more polymerized water soluble monomers or salts thereof, selected from the group consisting of
(meth)acrylic acid,
acrylamido-2-methylpropanesulphonic acid,
styrenesulphonic acid,
vinylsulphonic acid,
(meth)allylsulphonic acid,
vinylphosphonic acid,
maleic anhydride,
itaconic acid,
dimethyldiallylammonium chloride,
quaternized dimethylaminoethyl methacrylate,
(meth)acrylamidopropyltrimethylammonium chloride,
methylvinylimidazolium chloride,
a hydrophobic vinyl monomer of formula (A): 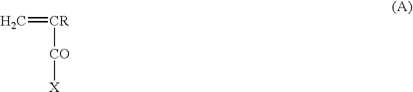
in which:
R is at least one of H, —CH₃, —C₂H₅ or —C₃H₇,
X is at least one of:
an alkyl oxide of formula —OR′ in which R′ is a linear or branched, saturated or unsaturated hydrocarbon radical containing from 1 to 6 carbon atoms, substituted with at least one of a sulphonic group (—SO₃⁻); a sulphate group (—SO₄⁻); a phosphate group (—PO₄H₂⁻); or a quaternary amine group (—N⁺R₁R₂R₃), wherein R₁, R₂ and R₃ are, independently of each other, a linear or branched, saturated or unsaturated hydrocarbon radical containing from 1 to 6 carbon atoms, wherein the sum of the number of carbon atoms of R′+R₁+R₂+R₃ does not exceed 6, wherein R′ is optionally substituted with at least one of a halogen atom selected from the group consisting of iodine, bromine, chlorine and fluorine; a hydroxyl group (—OH);
a primary amine group (—NH₂); a secondary amine group (—NHR₁); or a tertiary amine group (—NR₁R₂); wherein R₁, R₂ and R₃ are, independently of each other, a linear or branched, saturated or unsaturated hydrocarbon radical containing from 1 to 6 carbon atoms, and wherein the sum of the carbon atoms of R′+R₁+R₂+R₃ does not exceed 6; or
an —NH₂, —NHR′ or —NR′R″ group in which R′ and R″ are, independently of each other, linear or branched, saturated or unsaturated hydrocarbon radicals containing from 1 to 6 carbon atoms, wherein the total number of carbon atoms of R′+R″ does not exceed 6, wherein at least one of R′ or R″ may optionally be substituted with at least one of a sulphonic group (—SO₃⁻); a sulphate group (—SO₄⁻); a phosphate group (—PO₄H₂⁻); or a quaternary amine group (—N⁺R₁R₂R₃), wherein R₁, R₂ and R₃ are, independently of each other, a linear or branched, saturated or unsaturated hydrocarbon radical containing from 1 to 6 carbon atoms, wherein the sum of the number of carbon atoms of R′+R₁+R₂+R₃ does not exceed 6, wherein the radicals R′ and R″ may be optionally substituted with at least one of a halogen atom selected from the group consisting of iodine, bromine, chlorine and fluorine; a hydroxyl group (—OH); a primary amine group (—NH₂); a secondary amine group (—NHR₁); or a tertiary amine group (—NR₁R₂); wherein R₁, R₂ and R₃ are, independently of each other, a linear or branched, saturated or unsaturated hydrocarbon radical containing from 1 to 6 carbon atoms, wherein the sum of the carbon atoms of R′+R″+R₁+R₂+R₃ does not exceed 6.

47. The process according to claim 41, wherein the amphiphilic copolymer comprises an ionic hydrophilic block comprising one or more polymerized acrylic acid monomers.

48. The process according to claim 41, wherein the hydrophilic block is nonionic.

49. The process according to claim 48, wherein the amphiphilic copolymer comprises a nonionic hydrophilic polymer block comprising one or more water-soluble monomers selected from the group consisting of
(meth)acrylamide,
N-vinylacetamide, N-methyl-N-vinylacetamide,
N-vinylformamide, -methyl-N-vinylformamide,
a vinyllactam comprising a cyclic alkyl group containing from 4 to 9 carbon atoms, N-vinylpyrrolidone, N-butyrolactam, N-vinyl-caprolactam,
a vinyl alcohol of formula CH₂═CHOH,
a glycidyl (meth)acrylate,
a hydrophobic vinyl monomer of formula (A): 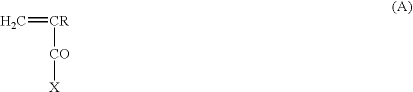
in which:
R is at least one of H, —CH₃, —C₂H₅ or —C₃H₇,
X is at least one of:
an alkyl oxide of formula —OR′ in which R′ is a linear or branched, saturated or unsaturated hydrocarbon radical containing from 1 to 6 carbon atoms, optionally substituted with at least one of a halogen atom selected from the group consisting of iodine, bromine, chlorine and fluorine; a hydroxyl group (—OH); a primary amine group (—NH₂); a secondary amine group (—NHR₁); or a tertiary amine group (—NR₁R₂); wherein R₁ and R₂ are, independently of each other, a linear or branched, saturated or unsaturated hydrocarbon radical containing from 1 to 6 carbon atoms, and wherein the sum of the carbon atoms of R′+R₁+R₂ does not exceed 6; or
an —NH₂, —NHR′ or —NR′R″ group in which R′ and R″ are, independently of each other, linear or branched, saturated or unsaturated hydrocarbon radicals containing from 1 to 6 carbon atoms, wherein the total number of carbon atoms of R′+R″ does not exceed 6, wherein R′ and R″ may optionally be substituted with at least one of a halogen atom selected from the group consisting of iodine, bromine, chlorine and fluorine; a hydroxyl group (—OH);
a primary amine group (—NH₂); a secondary amine group (—NHR₁); or a tertiary amine group (—NR₁R₂); wherein R₁ and R₂ are, independently of each other, a linear or branched, saturated or unsaturated hydrocarbon radical containing from 1 to 6 carbon atoms, and wherein the sum of the carbon atoms of R′+R″+R₁+R₂ does not exceed 6.

50. The process according to claim 49, wherein the nonionic hydrophilic polymer block is at least one of polyethylene oxide or polyvinyl-pyrrolidone.

51. The process according to claim 41, wherein the hydrophobic polymer block is at least one selected from the group consisting of polystyrene, poly(tert butylstyrene), poly(methyl methacrylate), poly(ethyl acrylate), poly(butyl methacrylate), a polycaprolactone, a polycaprolactam, a polydimethylsiloxane, a poly(C₃-C₆ alkylene oxide), poly(aspartic acid), poly(lactic acid), poly(glycolic acid), polyleucine, polybutadiene, polyethylene, polypropylene and polybutylene.

52. The process according to claim 41, wherein the block amphiphilic copolymer is at least one selected from the group consisting of:
polystyrene/polyoxyethylene,
polymethyl methacrylate/polyoxyethylene,
polybutyl methacrylate/polyoxyethylene,
polyoxybutylene/polyoxyethylene,
polycaprolactone/polyoxyethylene,
polyethylene/polyoxyethylene, and
polyoxyethylene/polyoxybutylene/polyoxyethylene.

53. The process according to claim 41, wherein the lipophilic compound is at least one selected from the group consisting of an emollient, an antiinflammatory agent, an antibacterial agent, an antifungal agent, an antiviral agent, an antiseborrhoeic agent, an antiacne agent, a keratolytic agent, an antihistamine, an anaesthetic, a cicatrizing agent, a pigmentation modifier, a tanning accelerator, an artificial tanning agent, a liporegulator, an anti-ageing agent, an anti-wrinkle agent, a refreshing agent, a vascular protector, an insect repellant, a deodorant, an antidandruff agent, an agent for preventing hair loss, an essential oil, a sunscreen, an antioxidant, a free-radical scavenger and a moisturizer.

54. The process according to claim 41, wherein the lipophilic compound is at least one selected from the group consisting of a vitamin, vitamin A (retinol), an ester of vitamin A, vitamin E, an ester of vitamin E, tocopheryl acetate, vitamin D, a derivative of vitamin D, vitamin F, a derivative of vitamin F, a carotene, β-carotene, a derivative of β-carotene, lycopene, and salicylic acid derivatives.

55. The process according to claim 54, wherein the lipophilic compound is at least one salicylic acid derivative selected from the group consisting of 5-n-octanoylsalicylic, 5-n-decanoylsalicylic, 5-n-dodecanoylsalicylic, 5-n-octyisalicylic, 5-n-heptyloxysalicylic, 4-n-heptyloxysalicylic, 5-tert-octylsalicylic, 3-tert-butyl-5-methylsalicylic, 3-tert-butyl-6-methyl-salicylic, 3,5-diisopropylsalicylic, 5-butoxysalicylic, 5-octyloxysalicylic, 5-propanoylsalicylic, 5-n-hexa-decanoylsalicylic, 5-n-oleoylsalicylic and 5-benzoylsalicylic acid, monovalent salts thereof, divalent salts thereof, and mixtures thereof.

56. The process according to claim 53, wherein the lipophilic compound is a sunscreen selected from the group consisting of an anthranilate; a cinnamic derivative; a dibenzoylmethane derivative; a salicylic derivative; a camphor derivative; a triazine derivative; a 1,3,5-triazine derivative; a benzophenone derivative; a β,β′-diphenylacrylate derivative; a benzotriazole derivative; a benzalmalonate derivative; a benzimidazole derivative; an imidazoline; a bis-benzazolyl derivative; a p-aminobenzoic acid (PABA) derivative; a methylenebis(hydroxyphenylbenzotriazole) derivative; a screening polymer; a screening silicone; a dimer derived from α-alkylstyrene; a 4,4-diarylbutadiene, and mixtures thereof.

57. The process according to claim 56, wherein the lipophilic compound is at least one 1,3,5-triazine derivative selected from the group consisting of:
2-[(p-(tert-butylamido)anilino]-4,6-bis[(p-(2′-ethylhexyl-1′-oxycarbonyl)anilino]-1,3,5-triazine;
2,4,6-tris[p′-(2′-ethylhexyl-1′-oxycarbonyl)anilino]-1,3,5-triazine;
2,4-bis {[4-2-ethylhexyloxy]-2-hydroxy]phenyl}-6-(4-methoxyphenyl)-1,3,5-triazine;
2,4,6-tris(diisobutyl 4′-aminobenzalmalonate)-s-triazine;
and mixtures thereof.

58. The process according to claim 56, wherein the lipophilic compound is a butylmethoxydibenzoylmethane.

59. The process according to claim 41, wherein the lipophilic compound is at least one selected from the group consisting of
a dehydroepiandrosterone, a biological precursor of dehydroepiandrosterone, a derivative, a plant sterol, and esters thereof, with the exception of cholesterol and esters thereof;
a pentacyclic triterpene acid, ursolic acid, oleanolic acid,
a hydroxystilbene,
an isoflavonoid,
an aminophenol derivative of formula (III) 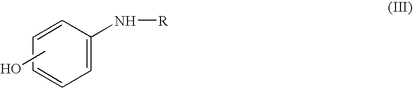
in which R is a radical corresponding to one of formula (i), (ii) or (iii)
—CO—NR₁R₂ (i)
—CO—O—R₃ (ii)
—SO₂—R₃ (iii)
wherein
R₁ is a hydrogen atom or a linear or branched, saturated or unsaturated, optionally hydroxylated C₁₋₆ alkyl radical, R₂ is a hydrogen atom or saturated or unsaturated, linear, cyclic or branched, C₁₂ to C₃₀ optionally hydroxylated alkyl radical, and R₃ is at least one of a saturated or unsaturated, linear, branched or cyclic C₁₂ to C₃₀ alkyl radical, or a optionally hydroxylated fused polycyclic radical.

60. The process according to claim 59, wherein the lipophilic compound comprises at least one selected from the group consisting of dehydroepiandrosterone (DHEA), DHEA sulphate, 7-hydroxy-DHEA, 7-keto-DHEA, prednisolone, prednisone, progesterone, pregnenolone, testosterone, diosgenin, hecogenin, ursolic acid, oleanolic acid, resveratrol, N-cholesteryloxycarbonyl-4-aminophenol, and an iso-flavonoid having a solubility in water at room temperature (25° C.) of less than 0.01%.

61. A cosmetic composition comprising at least one aqueous phase, at least one lipophilic compound and at least one block amphiphilic copolymer comprising at least one of an ionic or a nonionic hydrophilic polymer block and at least one hydrophobic polymer block wherein the hydrophobic polymer block comprises polymerized monomers units of at least one selected from the group consisting of:
styrene, derivatives thereof, 4-butylstyrene,
vinyl acetate of formula CH₂═CH—OCOCH₃, a vinyl ether of formula CH₂═CHOR in which R is a linear or branched, saturated or unsaturated hydrocarbon radical containing from 1 to 6 carbon atoms,
acrylonitrile,
vinyl chloride, vinylidene chloride,
caprolactone, caprolactam,
an alkene, ethylene, propylene, butylene, butadiene,
an alkylene oxide containing at least 4 carbon atoms, an alkylene oxide containing from 4 to 6 carbon atoms,
a silicon-containing polymerizable monomer capable of forming a polysiloxane,
a hydrophobic vinyl monomer of formula (A): 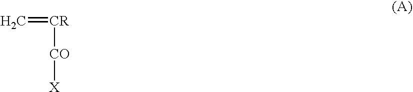
in which:
R is at least one of H, —CH₃, —C₂H₅ or —C₃H₇,
X is at least one of:
an alkyl oxide of formula —OR′ in which R′ is a linear or branched, saturated or unsaturated hydrocarbon-based radical containing from 1 to 22 carbon atoms, optionally substituted with at least one of a halogen atom selected from the group consisting of iodine, bromine, chlorine and fluorine; a sulphonic group (—SO₃⁻); a sulphate group (—SO₄⁻); a phosphate group (—PO₄H₂⁻); a hydroxyl group (—OH); a primary amine group (—NH₂); a secondary amine group (—NHR₁), a tertiary amine group (—NH₁R₂) or a quaternary amine group (—N⁺R₁R₂R₃) wherein R₁, R₂ and R₃ are, independently of each other, a linear or branched, saturated or unsaturated hydrocarbon based radical containing from 1 to 22 carbon atoms, and wherein the sum of the carbon atoms of R′+R₁+R₂+R₃ does not exceed 22, or
an —NH₂, —NHR′ or —NR′R″ group in which R′ and R″ are, independently of each other, linear or branched, saturated or unsaturated hydrocarbon radicals containing from 1 to 22 carbon atoms, wherein the total number of carbon atoms of R′+R″ does not exceed 22, wherein R′ and R″ may optionally be substituted with at least one of a halogen atom selected from the group consisting of iodine, bromine, chlorine and fluorine; a hydroxyl group (—OH); a sulphonic group (—SO₃⁻); a sulphate group (—SO₄⁻); a phosphate group (—PO₄H₂⁻); a primary amine group (—NH₂); a secondary amine group (—NHR₁); a tertiary amine group (—NR₁R₂) or a quaternary amine group (—N+R₁R₂R₃); wherein R₁, R₂ and R₃ are, independently of each other, a linear or branched, saturated or unsaturated hydrocarbon radical containing from 1 to 22 carbon atoms, wherein the sum of the carbon atoms of R′+R″+R₁+R₂+R₃ does not exceed 22, wherein R′ and R″ may optionally be perfluoroalkyl radicals having from 1 to 18 carbon atoms.

62. The composition according to claim 61, wherein the block amphiphilic copolymer is the sole solvent for the lipophilic compound in the composition.

63. The composition according to claim 61, wherein the molecular weight of the block copolymer is between 1,000 and 100,000.

64. The composition according to claim 61, wherein the weight ratio of the ionic or nonionic hydrophilic polymer block to the hydrophobic polymer block is between 1/100 and 50/1.

65. The composition according to claim 61, wherein the weight concentration ratio between the lipophilic compound and the block copolymer is between 0.005 and 0.5.

66. The composition according to claim 61, wherein the hydrophobic polymer block comprises one or more polymerized monomer units selected from the group consisting of methyl methacrylate, ethyl methacrylate, n-butyl (meth)acrylate, tert-butyl (meth)acrylate, cyclohexyl acrylate, isobornyl acrylate, 2-ethylhexyl acrylate, ethyl perfluorooctyl acrylate and trifluoromethyl (meth)acrylate.

67. The composition according to claim 61, wherein the hydrophilic block is nonionic.

68. The composition according to claim 67, wherein the nonionic hydrophilic polymer block comprises one or more polymerized water-soluble monomers selected from the group consisting of
(meth)acrylamide,
N-vinylacetamide, N-methyl-N-vinylacetamide,
N-vinylformamide, N-methyl-N-vinylformamide,
a N-vinyllactam comprising a cyclic alkyl group containing from 4 to 9 carbon atoms, N-vinylpyrrolidone, N-butyrolactam, N-vinyl-caprolactam,
a vinyl alcohol of formula CH₂═CHOH,
a glycidyl (meth)acrylate,
a water-soluble vinyl monomer of formula (A) below: 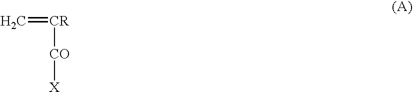
in which:
R is at least one of H, —CH₃, —C₂H₅ or —C₃H₇,
X is at least one of:
an alkyl oxide of formula —OR′ in which R′ is a linear or branched, saturated or unsaturated hydrocarbon radical containing from 1 to 6 carbon atoms, optionally substituted with at least one of a halogen atom selected from the group consisting of iodine, bromine, chlorine and fluorine; a hydroxyl group (—OH); a primary amine group (—NH₂); a secondary amine group (—NHR₁); or a tertiary amine group (—NR₁R₂); wherein R₁ and R₂ are, independently of each other, a linear or branched, saturated or unsaturated hydrocarbon radical containing from 1 to 6 carbon atoms, and wherein the sum of the carbon atoms of R′+R₁+R₂ does not exceed 6; or
an —NH₂, —NHR′ and —NR′R″ group in which R′ and R″ are, independently of each other, linear or branched, saturated or unsaturated hydrocarbon radicals containing from 1 to 6 carbon atoms, wherein the total number of carbon atoms of R′+R″ does not exceed 6, wherein R′ and R″ may optionally be substituted with at least one of a halogen atom selected from the group consisting of iodine, bromine, chlorine and fluorine; a hydroxyl group (—OH);
a primary amine group (—NH₂); a secondary amine group (—NHR₁); and a tertiary amine group (—NR₁R₂); wherein R₁ and R₂ are, independently of each other, a linear or branched, saturated or unsaturated hydrocarbon radical containing from 1 to 6 carbon atoms, and wherein the sum of the carbon atoms of R′+R″+R₁+R₂ does not exceed 6.

69. The composition according to claim 61, wherein the amphiphilic copolymer comprises at least one nonionic hydrophilic polymer block one selected from the group consisting of polyethylene oxide and polyvinylpyrrolidone.

70. The composition according to claim 51, wherein the hydrophobic polymer block is at least one selected from the group consisting of polystyrene, poly(tert butylstyrene), poly(methyl methacrylate), poly(ethyl acrylate), poly(butyl methacrylate), a polycaprolactone, a polycaprolactam, a polydimethylsiloxane, a poly(C₃-C₆ alkylene oxide), poly(aspartic acid), poly(lactic acid), poly(glycolic acid), polyleucine, polybutadiene, polyethylene, polypropylene and polybutylene.

71. The composition according to claim 61, wherein the block amphiphilic copolymer is at least one selected from the group consisting of:
polystyrene/polyoxyethylene,
polymethyl methacrylate/polyoxyethylene,
polybutyl methacrylate/polyoxyethylene,
polyoxybutylene/polyoxyethylene,
polycaprolactone/polyoxyethylene,
polyethylene/polyoxyethylene, and
polyoxyethylene/polyoxybutylene/polyoxyethylene.

72. The composition according to claim 61, wherein the lipophilic compound is at least one selected from the group consisting of an emollient, an anti-inflammatory agent, an antibacterial agent, an antifungal agent, an antiviral agent, an anti-seborrhoeic agent, an antiacne agent, a keratolytic agent, an antihistamine, an anaesthetic, a cicatrizing agent, a pigmentation modifier, a tanning accelerator, an artificial tanning agent, a liporegulator, an anti-ageing agent, an anti-wrinkle agent, a refreshing agent, a vascular protector, an insect repellant, a deodorant, an antidandruff agent, an agent for preventing hair loss, an essential oil, a fragrance, a sunscreen, an antioxidant, a free-radical scavenger, a moisturizer and a vitamin.

73. The composition according to claim 61, wherein the lipophilic compound is at least one selected from the group consisting of a vitamin, vitamin A (retinol), an ester of vitamin A, vitamin E, an ester of Vitamin E, tocopheryl acetate, vitamin D, a derivative of vitamin D, vitamin F, a derivative of vitamin F, a carotene, β-carotene, a derivative of β-carotene, lycopene, and a salicylic acid derivative.

74. The composition according to claim 73, comprising at least one salicylic acid derivative selected from the group consisting of 5-n-octanoylsalicylic, 5-n-decanoylsalicylic, 5-n-dodecanoylsalicylic, 5-n-octylsalicylic, 5-n-heptyloxysalicylic, 4-n-heptyloxysalicylic, 5-tert-octylsalicylic, 3-tert-butyl-5-methylsalicylic, 3-tert-butyl-6-methyl-salicylic, 3,5-diisopropylsalicylic, 5-butoxysalicylic, 5-octyloxysalicylic, 5-propanoylsalicylic, 5-n-hexa-decanoylsalicylic, 5-n-oleoylsalicylic, a 5-benzoylsalicylic acid derivative, a monovalent salt thereof, divalent salt thereof, and mixtures thereof.

75. The composition according to claim 72, comprising at least one sunscreen selected from the group consisting of an anthranilate; a cinnamic derivative; a dibenzoylmethane derivative; a salicylic derivative; a camphor derivative; a triazine derivative; a 1,3,5-triazine derivative; a benzophenone derivative; a β,β′-diphenylacrylate derivative; a benzotriazole derivative; a benzalmalonate derivative; a benzimidazole derivative; an imidazoline; a bis-benzazolyl derivative; a p-aminobenzoic acid (PABA) derivative; a methylenebis(hydroxyphenylbenzotriazole) derivative; a screening polymer, a screening silicone; a dimer derived from α-alkylstyrene; a 4,4-diarylbutadiene, and mixtures thereof.

76. The composition according to claim 75, comprising a 1,3,5-triazine derivative selected from the group consisting of:
2-[(p-(tert-butylamido)anilino]-4,6-bis[(p-(2′-ethylhexyl-1′-oxycarbonyl)anilino]-1,3,5-triazine,
2,4,6-tris[p′-(2′-ethylhexyl-1′-oxycarbonyl)anilino]-1,3,5-triazine,
2,4-bis {[4-2-ethylhexyloxy]-2-hydroxy]phenyl)-6-(4-methoxyphenyl)-1,3,5-triazine,
2,4,6-tris(diisobutyl 4′-aminobenzalmalonate)-s-triazine, and mixtures thereof.

77. The composition according to claim 76, comprising a butylmethoxydibenzoylmethane.

78. The composition according to claim 61, wherein the lipophilic compound is at least one selected from the group consisting of
a dehydroepiandrosterone (DHEA), a biological precursor of DHEA, a derivative of DHEA, an ester of DHEA, a plant sterol, a phytosterol, a sitosterol, and esters thereof, with the exception of cholesterol and esters thereof,
a pentacyclic triterpene acid, ursolic acid, oleanolic acid,
an hydroxystilbene,
an isoflavonoids,
an aminophenol derivative of formula (III) 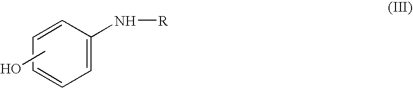
in which R is a radical corresponding to one of the formula (i), (ii) or (iii)
—CO—NR₁R₂ (i)
—CO—O—R₃ (ii)
—SO₂—R₃ (iii)
in which:
R₁ is a hydrogen atom or a linear or branched, saturated or unsaturated, optionally hydroxylated C₁₋₆ alkyl radical, R₂ is a hydrogen atom or a saturated or unsaturated, linear, cyclic or branched, C₁₂ to C₃₀ optionally hydroxylated alkyl radical, and R₃ is at least one of a saturated or unsaturated, linear, branched or cyclic C₁₂ to C₃₀ alkyl radical, or an optionally hydroxylated fused polycyclic radical.

79. The composition according to claim 78, wherein the lipophilic compound is at least one selected from the group consisting of dehydroepiandrosterone (DHEA), DHEA sulphate, 7-hydroxy-DHEA, 7-keto-DHEA, prednisolone, prednisone, progesterone, pregnenolone, testosterone, diosgenin, hecogenin, ursolic acid, oleanolic acid, resveratrol, N-cholesteryloxycarbonyl-4-aminophenol, and an iso-flavonoid having solubility in water at room temperature (25° C.) is less than 0.01%.

80. The composition according to claim 61, further comprising
one or more formulation adjuvants selected from the group consisting of a fatty substance, an organic solvent, a thickener, an opacifier, a stabilizer, an antifoam, a preserving agent, a filler, a sequestering agent, a propellant and a dye.

81. The process of claim 41, wherein the hydrophobic polymer block comprises polymerized monomer units of the hydrophobic vinyl monomer of formula (A) wherein R′ and R″ have from 1 to 18 carbon atoms.

82. The process as claimed in claim 44, wherein the weight concentration ratio between the lipophilic compound and the block amphiphilic copolymer is between 0.005 and 0.2.

83. The process according to claim 53, wherein the lipophilic compound is at least one essential oil selected from the group consisting of eucalyptus oil, lavandin oil, lavender oil, vetiver oil, Litsea cubeba oil, lemon oil, sandlewood oil, rosemary oil, camomile oil, savory oil, nutmeg oil, cinnamon oil, hyssop oil, caraway oil, orange oil, geraniol oil and cade oil.

84. The composition according to claim 65, wherein the weight concentration ratio between the lipophilic compound and the block copolymer is between 0.005 and 0.02.

85. The composition according to claim 72, comprising at least one essential oil selected from the group consisting of eucalyptus oil, lavandin oil, lavender oil, vetiver oil, Litsea cubeba oil, lemon oil, sandlewood oil, rosemary oil, camomile oil, savory oil, nutmeg oil, cinnamon oil, hyssop oil, caraway oil, orange oil, geraniol oil and cade oil.

86. The process of claim 41, wherein the lipophilic compound, the block amphiphilic copolymer, and water are mixed concurrently.
